(19) United States
(12) Patent Application Publication
(10) Pub. No.: US 2010/0036350 A1
(43) Pub. Date: Feb. 11, 2010

(54) HYGIENIC BAND EQUIPPED WITH A MAGNET

(76) Inventor: Rin Heo, Seoul (KR)

Correspondence Address: ROTHWELL, FIGG, ERNST & MANBECK, P.C., 1425 K STREET, N.W., SUITE 800, WASHINGTON, DC, 20005, US

(21) Appl. No.: 12/373,123

(22) PCT Filed: Jul. 11, 2006
(86) PCT No.: PCT/KR2006/002720

§ 371 Date: Jun. 17, 2009

(51) Int. Cl. A61F 13/45

(52) U.S. Cl. 604/385.01

(57) ABSTRACT

A hygienic band with magnets attached or built in is disclosed. More particularly, the present invention is related to a hygienic band in which a plurality of weak magnets having magnetism are compactly attached in accordance with a predetermined principle, so as to not only solve or relieve premenstrual syndrome (PMS) including nausea, displeasure, and pains, but also prevent release of menstrual blood in a clot state by loosening clots of the menstrual blood, thereby relieving menstrual pains at each part of the body.

### TECHNICAL FIELD

The present invention relates to a hygienic band with magnets attached or built in, and more particularly to a hygienic band in which a plurality of weak magnets are compactly attached or inserted, so as to not only solve premenstrual syndrome (PMS) by promoting blood circulation around the vulva and the womb, but also relieve menstrual pain by preventing release of menstrual blood in a clot state by helping the menstrual blood clots get loosened.

### BACKGROUND ART

In general, menstruation of women means bleeding from the endometrium occurring periodically. A hygienic band is used to hygienically dispose of the menstrual blood. The hygienic band generally comprises a waterproof outer cover disposed at a bottom layer to prevent permeation of the menstrual blood, an absorbent layer for absorbing the menstrual blood, an unwoven fabric layer made of a soft material for direct contact with a skin, and a dual-side adhesive tape disposed at the lowermost layer for fixation to underclothes. When a user wears the general hygienic band comprising in which unwoven fabric layer, the absorbent layer, the waterproof outer cover are accumulated in this order, the menstrual blood released from the body is passed through the unwoven fabric layer, soaked into the absorbent layer and then obstructed by the waterproof outer cover. Thus, the hygienic band prevents the menstrual blood from flowing out and staining the underclothes.

However, such a general hygienic band as described above functions only as a cover for preventing leakage of the menstrual blood. In other words, the general hygienic band does not have any other functions such as relieving menstrual pain or loosening clots of the menstrual blood when the menstrual blood is released in the clot state. Therefore, most of menstruating women have been suffering from the menstrual pain. Nevertheless, they often endure the pain even without taking pills like a painkiller because the menstrual pain is not a bacterial symptom that requires medication. Moreover, the clot of menstrual blood brings unpleasant feelings to whom wearing the hygienic band, thereby deteriorating wearing comfort of the hygienic band.

Meanwhile, it is widely known that magnetic poles (N pole and S pole) have various positive effects for a human body, including promotion of blood circulation and metabolism, maintenance and recover of functions of each part of the human body, and relief of pains. Accordingly, various products such as a magnet bracelet or belt, a magnet mat, and magnet underclothes that utilize the above effects of the magnet have been developed and put on the market.

### DISCLOSURE OF INVENTION

### Technical Problem

However, there has not been suggested a hygienic band applying a magnet so far. During menstruation, muscles in the womb shrink to discharge the menstrual blood and this causes excessive secretion of prostaglandin which is a hormone relevant to this operation. Since bloodstreams to the other peripheral parts except for the womb are hindered by the excessive prostaglandin, oxygen may not be favorably and sufficiently supplied to terminal organs, thereby incurring ischemia at the terminal organs and dropping the body temperature. Also, the muscle shrinkage and the lack of oxygen cause the menstrual pain. If the magnet having the positive effects is applied to the hygienic band, the blood circulation will be promoted by the magnetism and oxygen will be sufficiently supplied into blood vessels, consequently preventing the drop of body temperature and relieving the menstrual pain. This theory has been proved through clinical experiments performed with a number of impregnable women.

### Technical Solution

Therefore, the present invention has been made in view of the above problems, and it is an object of the present invention to provide a hygienic band attached or built in with magnets which are known to have various effects such as promoting blood circulation and metabolism and relieving pain, the hygienic band in which a plurality of magnets having a coin or thin bar shape or rubber magnets are attached compactly in such a manner that N poles and S poles are alternately arranged one by one, so as to not only relieve premenstrual syndrome (PMS) including nausea, displeasure and pains, but also prevent release of menstrual blood in a clot state by loosening clots of the menstrual blood, thereby relieving menstrual pains at each part of the body.

### Advantageous Effects

According to the present invention, a plurality of magnets having various effects such as promotion of blood circulation and metabolism and relief of pain, are manufactured in the form of a coin or a thin bar and attached or built in a hygienic band in such a manner that N poles and S poles are alternately arranged one by one. Magnetism is maximally enhanced through the above arrangement, and therefore PMS and menstrual pain can be relieved. Also, by loosening clots of menstrual blood released, an unpleasant feeling can be reduced, thereby improving wearing comfort of the hygienic band.

### DESCRIPTION OF DRAWINGS

The accompanying drawings illustrate preferred embodiments of the present invention. This invention may, however, be embodied in different forms and should not be construed as limited to the embodiments set forth in the drawings.

The above and other objects, features and other advantages of the present invention will be more clearly understood from the following detailed description taken in conjunction with the accompanying drawings, in which:

FIG. 1 is a perspective view of a hygienic band according to an embodiment of the present invention;

FIG. 2 is a plan view showing the arrangement of magnets attached to or built in the hygienic band;

FIG. 3 is another plan view showing the arrangement of magnets attached to or built in the hygienic band;

FIG. 4 is a longitudinal sectional view of FIG. 2; and

FIG. 5 is a sectional view of an insertion-type hygienic band according to another embodiment of the present invention.

### BEST MODE

Hereinafter, a certain embodiment of the present invention will be described in detail with reference to the accompanying drawings.

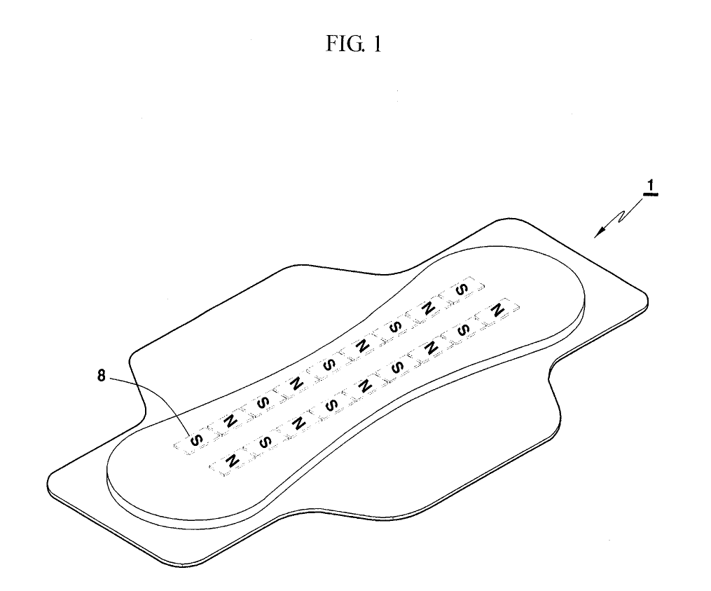

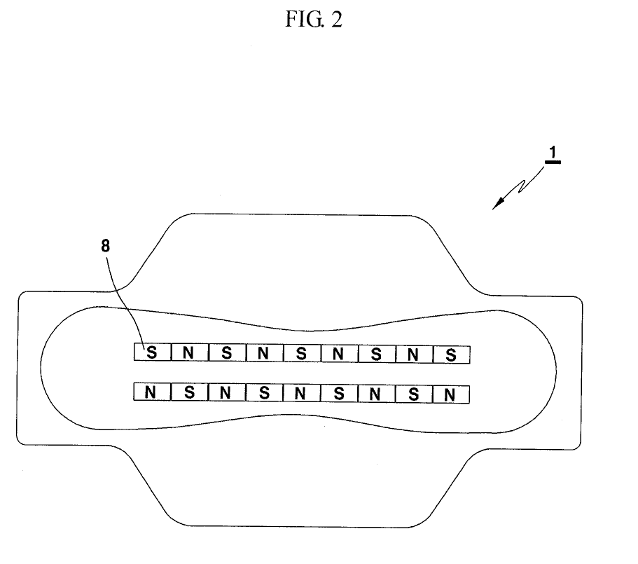

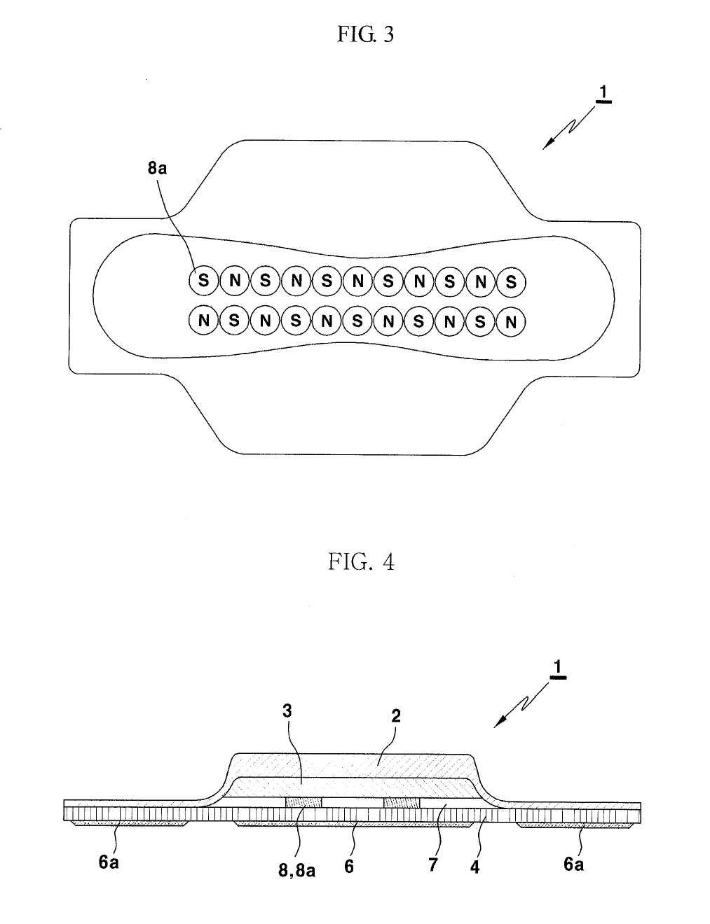

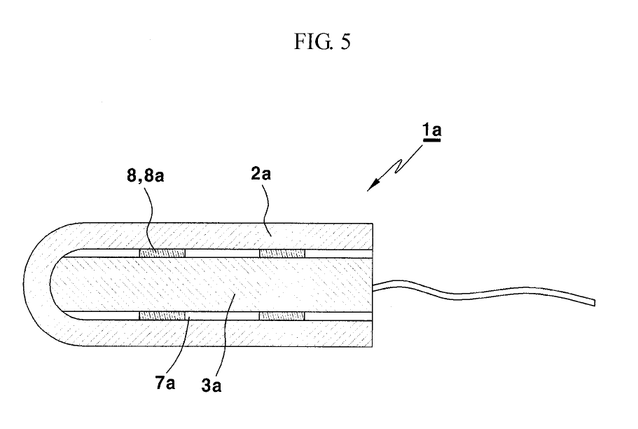

FIG. 1 is a perspective view of a hygienic band according to an embodiment of the present invention. FIG. 2 is a plan view showing the arrangement of magnets attached to or built in the hygienic band and FIG. 3 is another plan view showing the arrangement of magnets attached to or built in the hygienic band. FIG. 4 is a longitudinal sectional view of FIG. 3. As shown in FIGS. 1 through 3, the hygienic band 1 according to the present invention comprises an unwoven fabric layer 2 made of a soft material for direct contact with a skin, an absorbent layer 3 disposed under the unwoven fabric layer 2 to absorb menstrual blood, a waterproof outer cover 4 disposed under the absorbent layer 2 to prevent the menstrual blood from permeating through and leaking to the outside, and dual-side adhesive tapes 6 and 6a attached to a lower surface of wings 5 and 5a extended from the waterproof outer cover 4, respectively. A dedicated space 7 is formed between the absorbent layer 3 and the waterproof outer cover 4, and a plurality of magnets 8 in the form of a thin bar are compactly built in the space 7. More specifically, the magnets 8 are arranged in a pair of rows in such a manner that the respective magnet pairs are arranged to have opposite poles alternately, for example, in order of N-S, S-N, N-S, S-N and then the same. Coin-type magnets 8a may be used instead of the thin-bar magnets 8. Also, the thin-bar magnets 8 and the coin magnets 8a may be implemented by rubber magnets.

### MODE FOR INVENTION

FIG. 4 shows an insertion-type hygienic band, which is called a ‘tampon’, according to another embodiment of the present invention. The insertion-type hygienic band 1a comprises an unwoven fabric layer 2a made of a soft material for direct contact with a skin, and an absorbent layer 3a disposed at an inner side of the unwoven fabric layer 2a to absorb menstrual blood. A dedicated space 7a is formed inside, that is, between the unwoven fabric layer 2a and the absorbent layer 3a so that a plurality of the magnets 8 in the form of a thin bar can be built in the space 7a. Here, the magnets 8 are arranged in a pair of rows in such a manner that the respective magnet pairs are arranged to have opposite poles alternately, for example, in order of N-S, S-N, N-S, S-N and then the same. The coin-type magnets 8a may be used instead of the thin-bar magnets 8. Also, the thin-bar magnets 8 and the coin magnets 8a may be implemented by rubber magnets.

### INDUSTRIAL APPLICABILITY

Using the dual-side adhesive tapes 6 and 6a attached to the waterproof outer cover 4 and the wings 5 and 5a extended from the waterproof outer cover 4, a user attaches the above-structured hygienic band to an inside of the underclothes and puts on the underclothes. Here, since the plurality of thin-bar magnets 8 or the coin magnets 8a inserted in the space 7 between the absorbent layer 3 and the waterproof outer cover 4 are formed into thin slices, wearing comfort of the hygienic band is not deteriorated in comparison with conventional hygienic bands. In addition, as well as preventing leakage of the menstrual blood during the wearing which is the function of general conventional hygienic bands, the hygienic band according to the present invention is further capable of reducing the PMS and the menstrual pain by the magnets 8 and 8a inserted in the space 7 between the absorbent layer 3 and the waterproof outer cover 4 because of magnetism of the magnets 8 and 8a that promotes blood circulation and supply of oxygen into blood vessels. Furthermore, the magnets 8 and 8a helps loosen the menstrual blood clots so that the wearing comfort can be improved. These effects are proved through experiments performed with a number of impregnable women.

As to the magnet poles arrangement, more specifically, if the first pair of the magnet rows are arranged in order of N pole and S pole, the second pair are then arranged in order of S pole and N pole, and the third pair are arranged in order of N pole and S pole again to have the opposite poles between the neighboring rows. Thus, by compactly and repetitively arranging weak magnets having relatively weaker magnetism rather than strong magnets, the above effects, that is, promotion of blood circulation and oxygen supply, and relief of various PMS and the menstrual pain, can be expected.

Meanwhile, according to another embodiment, the principle of the present invention can be applied to the insertion-type hygienic band 1a, which is called a ‘tampon’ as shown in FIG. 4. Moreover, a pantyliner or a panty (not shown) may adopt the principle of the present invention for the same effects.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A hygienic band 1 comprising an unwoven fabric layer 2 made of a soft material for direct contact with the with a skin, an absorbent layer 3 disposed under the unwoven fabric layer 2 to absorb menstrual blood, a waterproof outer cover 4 disposed under the absorbent layer 2 to prevent the menstrual blood from flowing to the outside, and dual-side adhesive tapes 6 and 6a attached to a lower surface of wings 5 and 5a extended from the waterproof outer cover 4, the hygienic band 1 further comprising:
a dedicated space 7 formed between the absorbent layer 3 and the waterproof outer cover 4; and
a plurality of magnets 8 in the form of a thin bar, built in the space 7 and arranged in a pair of rows in such a manner that the respective magnet pairs are arranged to have opposite poles alternately, for example, in order of N-S, S-N, N-S, S-N and then the same.

2. The hygienic band according to claim 1, wherein a plurality of coin-type magnets 8a are built in the space 7 formed between the absorbent layer 3 and the waterproof outer cover 4, and arranged in a pair of rows in such a manner that the respective magnet pairs are arranged to have opposite poles alternately, for example, in order of N-S, S-N, N-S, S-N and then the same.

3. The hygienic band according to claim 1, wherein a plurality of rubber magnets are built in the space 7 formed between the absorbent layer 3 and the waterproof outer cover 4.

4. The hygienic band according to claim 1, comprising an insertion-type hygienic band 1a formed by an unwoven fabric layer 2a and an absorbent layer 3a disposed at an inner side of the unwoven fabric layer 2a, and further comprising:
a dedicated space 7a formed at the inner side between the unwoven fabric layer 2a and the absorbent layer 3a; and
a plurality of thin-bar magnets 8, coin magnets 8a, or rubber magnets attached or built in the space 7a.

5. The hygienic band according to claim 1, to which a panty or a pantyliner may be applied.

6. The hygienic band according to claim 2, wherein a plurality of rubber magnets are built in the space 7 formed between the absorbent layer 3 and the waterproof outer cover 4.
